(19) United States
(12) Patent Application Publication
(10) Pub. No.: US 2006/0123502 A1
(43) Pub. Date: Jun. 8, 2006

(54) Assay methods for identifying RE2-like antagonists, methods of use, and non-human transgenic animals

(76) Inventors: Andrew J. Murphy, Croton-on-Hudson, NY (US); Y. Gopi Shanker, New York, NY (US); Susan Croll-Kalish, Tarrytown, NY (US); Richard Torres, New York, NY (US)

Correspondence Address: REGENERON PHARMACEUTICALS, INC, 777 OLD SAW MILL RIVER ROAD, TARRYTOWN, NY, 10591, US

(21) Appl. No.: 11/292,149

(22) Filed: Nov. 30, 2005

Related U.S. Application Data
(60) Provisional application No. 60/633,261, filed on Dec. 3, 2004.

(51) Int. Cl. A01K 67/027; A61K 39/395; A61K 48/00

(52) U.S. Cl. 800/14; 800/16; 800/18; 514/44; 424/145.1

(57) ABSTRACT

Provided is a human RE2-L protein, as well as the encoding nucleic acid, methods for screening for agents capable of modulating RE2-L related activity and treating RE2-L-mediated conditions. Further provided are animal models useful for screening agents capable of ameliorating or reducing anxiety related disorders and obsessive-compulsive disorders.

### CROSS-REFERENCE TO RELATED APPLICATIONS

This application claims the benefit under 35 USC § 119(e) of U.S. Provisional application 60/633,261 filed 3 Dec. 2004, which application is herein specifically incorporated by reference in its entirety.

### BACKGROUND OF THE INVENTION

1. Field of the Invention

This invention is related to methods for identifying molecules capable of modulating RE2-Like (RE2-L) protein, therapeutic uses for such identified molecules, and animal models of human psychiatric disorders, particularly anxiety.

2. Description of Related Art

G-protein coupled receptors (GPCRs) are a class of integral membrane proteins, which contain seven hydrophobic transmembrane domains that span the cell membrane and form a cluster of anti-parallel alpha helices. GPCRs function in various physiological processes including vision, smell, neurotransmission, and hormonal responses. RE2-like protein (GPR101) is a GPCR mapping to a region of the X-chromosome frequently associated with X-linked mental retardation syndromes such as Sashi syndrome, Fragile X syndrome, and others.

Current treatments for anxiety disorders include tricyclic antidepressants (TCAs), selective serotonin reuptake inhibitors (SSRIs), and classical irreversible monoamine oxidase inhibitors (MAOIs). These are commonly used in the treatment in a broad range of anxiety disorders, including Generalized Anxiety Disorder (GAD) and Obsessive Compulsive Disorder (OCD). However, the poor tolerance of TCAs and the cardiac risks associated therewith, as well as the risks associated with conventional irreversible MAOIs, are limitations to their usefulness. Additionally, SSRIs have a slow onset of action, and are effective in less than two-thirds of patients.

### BRIEF SUMMARY OF THE INVENTION

Analysis of expression patterns of RE2-like using a LacZ knock-in revealed an especially intense localization of this gene to septum of the brain. The septum is a structure associated with many functions, most notably learning/memory and emotion. Experiments were conducted with knock-out animals having the endogenous RE2-L protein deleted (nucleic acid and amino acid sequence shown in SEQ ID NOs:1-2). These animals exhibited a phenotype of reduced stress relative to wild-type animals, identifying a role for RE-2-L protein in anxiety disorders. The discovery of the function of this protein allows for screening and therapeutic methods leading to the development of a novel therapeutics useful for reducing anxiety disorders.

Accordingly, in a first aspect, the invention provides screening methods for identifying agents capable of binding a human RE2-L protein. More specifically, the invention provides methods of identifying agents capable of inhibiting human RE2-L-mediated activity. Such agents are valuable as potential therapeutics for the treatment of psychiatric and neurological disorders such as the anxiety disorders, such as, obsessive-compulsive disorder (OCD), the pervasive developmental disabilities (PDDs) such as Asperger's Syndrome, Autism, and pervasive developmental disabilities not otherwise specified (PDD-NOS) and schizophrenia. The screening methods of the invention include in vitro and in vivo assays.

In one embodiment of an in vitro screening method of the invention, agents capable of binding the RE2-L protein are identified in a cell-based assay system. More specifically, cells expressing a RE2-L protein are contacted with a test compound, and the ability of the test compound to bind RE2-L or a fragment thereof is determined.

In another specific embodiment of the cell-based assay of the invention, the ability of a test compound to bind to RE2-L may be determined by a competitive binding assay. Accordingly, the ability of the test compound to competitively bind to RE2-L may be determined by obtaining cells expressing RE2-L, contacting the cells with one agent known to bind to RE2-L and a second agent whose ability to bind RE2-L is unknown, detecting the amount of binding of the first agent and comparing that amount with the amount of binding of the second agent. Binding of a compound to RE2-L may be determined in a number of ways known to the art, including for example, radioactive detection, fluorescence detection, chromogenic detection, mass spectroscopy, and plasmon resonance, or by detection of a biological response through measurement of Ca²⁺ ion flux, cAMP, IP₃, PIP₃ and transcription of reporter genes. In specific embodiments, RE2-L activity may be measured by measuring cAMP levels. In another embodiment, RE2-L activity may be measured using a reporter molecule, such as CRE, NFAT, SRE, or NF-κB.

In another embodiment, agents capable of binding a RE2-L protein are identified in a cell-free assay system. More specifically, a native or recombinant human RE2-L protein is contacted with a test compound, and the ability of the candidate compound to bind RE2-L is determined.

In another embodiment, agents capable of binding RE2-L or a fragment thereof are identified in an in vivo system. More specifically, a candidate agent or a control compound is administered to a suitable animal, and the effect on RE2-L-mediated activity is determined.

In a second aspect, screening methods are provided for identifying antagonists of the human RE2-L protein. The method of the invention includes in vitro screening assay, including cell-free and cell-based assays, as well as in vivo assays. More specifically, an antagonist of the human RE2-L protein is capable of inhibiting or blocking the activity and/or expression of human RE2-L. In a more specific embodiment, the agent capable of inhibiting RE2-L-mediated activity decreases the activity of human RE2-L, for example, a blocking antibody. In another more specific embodiment, the antagonist is capable of interfering with the expression of the gene encoding RE2-L, such as for example, an antisense or siRNA molecule. Generally, manipulation of RE2-L levels are believed to be therapeutically useful to alleviate obsessive compulsive disorders and the pervasive developmental disorders.

Agents identified by the method of the invention are potential therapeutics useful in the treatment of psychiatric, and some neurological disorders, such as, for example, anxiety, OCD, autism, PDD-NOS, Asperger's Syndrome, Tourette's Syndrome, and schizophrenia. Any suitable test known to the art for identifying and measuring psychiatric disorders in a test animal may be used to identify an agent useful in the treatment of psychiatric disorders in humans, such as the tests described below, e.g., the “elevated plus maze”, open field testing, light-dark exploration tests, social interaction testing, sensory testing, sensory gating testing, and/or quantification of animal freezing, defecations, rears and grooming (especially early or excessive).

In a third aspect, the invention embodies therapeutic methods for treating a RE2-L-mediated condition, comprising administering an agent capable of modulating RE2-L activity identified by a screening method of the invention to a subject in need thereof. In the therapeutic method of the invention, a RE2-L-mediated condition is a psychiatric disorder, such as schizophrenia and anxiety disorders including generalized anxiety disorder (GAD) and obsessive-compulsive disorder (OCD). A RE2-L-mediated condition may result from neurological impairment, which may be congenital or the result of trauma. In one embodiment, the agent administered is a compound identified through a screening method of the invention.

In a fourth aspect, the invention features pharmaceutical compositions useful for treatment of RE2-L-mediated psychiatric disorders and diseases, for diminishing anxiety and anxiety-related activity, or for modulating RE2-L-mediated motor activity, comprising an agent identified by a screening method of the invention.

In a fifth aspect, the invention features pharmaceutical compositions useful for treatment of RE2-L-mediated activity, including anxiety, in a subject suffering from or at risk thereof, comprising an agent identified by the screening method of the invention.

In a sixth aspect, the invention features a non-human transgenic animal comprising a modification of an endogenous RE2-L gene. As described more fully in U.S. Pat. No. 6,856,251, the transgenic animal of the invention is generated by targeting the endogenous RE2-L gene with a large targeting vector (LTVEC). In one embodiment of the transgenic animal of the invention, the animal is a knock-out wherein the RE2-L gene is altered or deleted such that the function of the endogenous RE2-L protein is reduced or ablated. In another embodiment, the transgenic animal is a knock-in animal modified to comprise an exogenous human RE2-L gene. Such transgenic animals are useful, for example, in identifying agents that diminish anxiety or modulate other activities that are mediated by the human RE2-L protein. Such transgenic animals are also useful in identifying agents that treat seizures and related disorders mediated by the human RE2-L protein.

In a related seventh aspect, the invention provides an animal model for use in identifying an agent capable of diminishing, reducing, and/or ameliorating psychiatric or neurological disorders. The RE2-L gene knock-out animals of the invention exhibit specific symptoms of compulsive motor activity, perseverative behavior, abnormal social behavior, and anxiety, and are thus useful in a variety of ways, including in vivo screening of potential therapeutic compounds capable of ameliorating, diminishing, or reducing GAD, autism, PDD-NOS, Asperger's Syndrome, and/or OCD. The effectiveness of the test agent may be determined by behavioral observation, such as for example observation of an animal in a elevated plus maze, light-dark exploration task, “Y” maze, social interaction tests, sensory tests, sensory gating tests, or open field.

Other objects and advantages will become apparent from a review of the ensuing detailed description.

### BRIEF DESCRIPTION OF THE FIGURES

FIGS. 1A-B shows mean latency times to escape in a water maze acquisition experiment for RE2-L knockout (KO) and wild-type (wt) mice. The results did not show a significant difference in delay in learning the location of the escape platform between KO and wt mice (FIG. 1A), or remembering the location once learned (FIG. 1B).

FIG. 2 is a bar graph showing that RE2-L KO animals spent more time in the open arms of the Elevated Plus Maze relative to wt animals.

FIG. 3 is a graph of basal activity of RE2-L in cells transfected with human RE2-L and a luciferase reporter (CRE-, SRE-, NFAT-, and NFκB-luciferase).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

Definitions

By the term “RE2-L-mediated condition” is meant a disease or condition associated with activity and/or expression of the RE2-L protein. More specifically, and as shown in the experimental results below, the RE2-L knock-out animals of the invention show that RE2-L is localized to several important regions of the brain, particularly in the septum of the brain. The septum is a structure associated with many functions, most notably, learning, memory, and emotion.

A “knock-out” animal is an animal generated from a mammalian cell that carries a genetic modification resulting from the insertion of a DNA construct targeted to a predetermined, specific chromosomal location that alters the function and/or expression of a gene that was at the site of the targeted chromosomal location. A transgenic “knock-in” animal is an animal generated from a mammalian cell that carries a genetic modification resulting from the insertion of a DNA construct targeted to a predetermined, specific chromosomal location that does not alter the function and/or expression of the gene at the site of the targeted chromosomal location. In both cases, the DNA construct may encode a reporter protein such as lacZ, protein tags, and proteins, including recombinases such as Cre and FLP.

General Description

The experiments described below identify the function of RE2-L as involved in the mediation of learning, memory, anxiety, and anxiety-related motor activity, the regulation of psychiatric disorders such as schizophrenia and anxiety disorders, and the modulation of motor activity. The instant invention provides, in part, screening assays for identification of molecules capable of inhibiting RE2-L-mediated activity, e.g., physiological events affected by the activation or inhibition of RE2-L.

Screening Assays

The present invention provides methods for identifying agents (e.g., candidate compounds or test compounds) that are capable of activating or inhibiting RE2-L-mediated activity or expression (collectively: modulating agents). Agents identified through the screening method of the invention are potential therapeutics for use in learning and/or memory disorders, as well as emotion disorders such as anxiety, nervous or compulsive motor activity, perseverative behaviors, and/or regulating psychiatric abnormalities such as anxiety disorders and schizophrenia.

Examples of agents to be tested by the screening methods of the invention include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, fully human, anti-idiotypic, chimeric, and single chain antibodies as well as Fab, F(ab′)₂, Fab expression library fragments, and epitope-binding fragments of antibodies). Further, agents or libraries of compounds may be presented, for example, in solution, on beads, chips, bacteria, spores, plasmids or phage.

In one embodiment, agents that bind RE2-L are identified in a cell-based assay system. In accordance with this embodiment, cells expressing a RE2-L protein or protein fragment are contacted with a candidate (or a control compound), and the ability of the candidate compound to bind RE2-L is determined.

The cell may be of prokaryotic origin (e.g., E. coli) or eukaryotic origin (e.g., yeast or mammalian). In specific embodiments, the cell is for example, a COS-7 cell, a 293 human embryonic kidney cell, a NIH 3T3 cell, or a Chinese hamster ovary (CHO) cell. Further, the cells may express a RE2-L protein or protein fragment endogenously or be genetically engineered to express a RE2-L protein or protein fragment. To identify ligands of RE2-L, cells expressing the receptor may be screened against a panel of know peptides utilizing a bioluminescent signal such as the aequorin luminescence assays (see, for example, Raddatz et al. (2000) J. Biol. Chem. 275:32452-32459 and Shan et al. (2000) J. Biol. Chem. 275:39482-39486, which publications are herein specifically incorporated by reference in their entireties). In these binding assays, the peptide to be tested is labeled. Cells expressing the RE2-L receptor are then incubated with labeled test compounds, in binding buffer, in cell culture dishes. To determine non-specific binding, unlabeled peptide may be added to the wells. After the incubation, bound and free peptides are separated and detection activity measured in each well.

The ability of the candidate compound to alter the activity of RE2-L can be determined by methods known to those of skill in the art, for example, by flow cytometry, a scintillation assay, immunoprecipitation or western blot analysis. For example, modulators of RE2-L-mediated activity may be identified using a biological readout in cells expressing a RE2-L protein or protein fragment. Agonists or antagonists are identified by incubating cells or cell fragments expressing RE2-L with test compound and measuring a biological response in these cells and in parallel cells or cell fragments not expressing RE2-L. An increased biological response in the cells or cell fragments expressing RE2-L compared to the parallel cells or cell fragments indicates the presence of an agonist in the test sample, whereas a decreased biological response indicates an antagonist.

In more specific embodiments, detection of binding and/or modulation of a test agent to a RE2-L protein may be accomplished by detecting a biological response, such as, for example, measuring Ca²⁺ ion flux, cAMP, IP₃, PIP₃ and transcription of reporter genes. Suitable reporter genes include endogenous genes as well as exogenous genes that are introduced into a cell by any of the standard methods familiar to the skilled artisan, such as transfection, electroporation, lipofection and viral infection. The invention further includes other end point assays to identify compounds that modulate (stimulate or inhibit) receptor activity, such as those associated with signal transduction.

The invention further provides a method of identifying an agent capable of modulating the expression of RE2-L, comprising (a) contacting a first population of cells expressing RE2-L with a candidate agent, (b) contacting a second population of cells expressing RE2-L with a control agent, and (c) comparing the level of RE2-L in the first and second populations of cells. In one embodiment, the level of RE2-L is greater in the first population of cells than in the second population of cells. In another embodiment, the level of RE2-L is less in the first population of cells than in the second population of cells. In a more specific embodiment, the level of RE2-L is determined by measurement of the corresponding mRNA.

In another embodiment, agents that bind RE2-L are identified in a cell-free assay system. In accordance with this embodiment, a RE2-L protein or protein fragment is contacted with a test (or control) compound and the ability of the test compound to bind RE2-L is determined. In vitro binding assays employ a mixture of components including a RE2-L protein or protein fragment, which may be part of a fusion product with another peptide or polypeptide, e.g., a tag for detection or anchoring, and a sample suspected of containing a natural RE2-L binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature that facilitates optimal binding. The mixture is incubated under conditions whereby the RE2-L protein binds the test compound. Incubation periods are chosen for optimal binding but are also minimized to facilitate rapid, high-throughput screening.

After incubation, the binding between the RE2-L protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radioactive emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that modulate (i.e., upregulate or downregulate) RE2-L-mediated activity or expression are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the RE2-L-mediated activity or expression is determined. Specifically, this method may be used to identify an agent capable of inhibiting anxiety, anxiety-related motor-activity, as well as nervous, compulsive motor activity, or to identify agents capable of modulating RE2-L-mediated psychiatric disorders and diseases. In addition, this method may specifically be used to identify an agent capable of treating seizures and related disorders.

Antibodies to Human RE2-L Protein and Ligands

The present invention provides for an antibody that specifically binds human RE2-L and is useful inhibiting RE2-L-mediated activity. According to the invention, a RE2-L protein, protein fragment, derivative or variant, may be used as an immunogen to generate immunospecific antibodies. Such immunogens can be isolated by any convenient means, including the methods described above. Antibodies may be blocking antibodies or activating antibodies and include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

Inhibitory Nucleic Acids

In addition to agents capable of inhibiting RE2-L activity, the methods of the invention encompass inhibition of RE2-L expression with nucleic acid molecules capable of interfering with or silencing RE2-L gene expression. In one embodiment, RE2-L expression is inhibited by RE2-L antisense nucleic acid comprises at least 6 to 200 nucleotides that are antisense to a gene or cDNA encoding RE2-L or a portion thereof. As used herein, a RE2-L “antisense” nucleic acid refers to a nucleic acid capable of hybridizing by virtue of some sequence complementarity to a portion of an RNA (preferably mRNA) encoding RE2-L. The antisense nucleic acid may be complementary to a coding and/or noncoding region of an mRNA encoding RE2-L. The oligonucleotides can be DNA or RNA or chimeric mixtures or derivatives or modified versions thereof, can be single- or double-stranded, and can be modified at the base moiety, sugar moiety, or phosphate backbone. The oligonucleotide may include other appended groups such as peptides; agents that facilitate transport across the cell membrane (see, e.g., Letsinger et al. (1989) Proc. Natl. Acad. Sci. USA 86:6553-6556) or blood-brain barrier (see, e.g., WO 89/10134,). Such antisense nucleic acids have utility as compounds that inhibit RE2-L expression, and can be used in the treatment of undesirable blood vessel formation.

In another embodiment, RE2-L may be inhibited with ribozymes or triple helix molecules which decrease RE2-L gene expression. Ribozyme molecules designed to catalytically cleave gene mRNA transcripts encoding RE2-L can be used to prevent translation of RE2-L mRNA and, therefore, expression of the gene product. (See, e.g., PCT International Publication W090/11364). Alternatively, the endogenous expression of RE2-L can be reduced by targeting deoxyribonucleotide sequences complementary to the regulatory region of the gene (i.e., the gene promoter and/or enhancers) to form triple helical structures that prevent transcription of RE2-L in target cells in the body (see, for example, Helene et al. (1992) Ann. N.Y. Acad. Sci., 660, 27-36).

In another embodiment, RE2-L is inhibited by a short interfering RNA (siRNA) through RNA interference (RNAi) or post-transcriptional gene silencing (PTGS) (see, for example, Ketting et al. (2001) Genes Develop. 15:2654-2659). siRNA molecules can target homologous mRNA molecules for destruction by cleaving the mRNA molecule within the region spanned by the siRNA molecule. Accordingly, siRNAs capable of targeting and cleaving homologous RE2-L mRNA are useful for inhibiting undesirable blood vessel formation.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105 ). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention that will be effective in the treatment of a RE2-L-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Therapeutic Methods and Combination Therapies

The invention is directed to therapeutically useful methods for treating any disease or condition which is improved, ameliorated, inhibited or prevented by modulation of RE2-L. Generally, inhibition of RE2-L may be useful to alleviate obsessive compulsive disorders. Activation of RE2-L is believed to be therapeutically useful in the treatment of autism or pervasive developmental disorders. In numerous embodiments, an agonist or antagonist of RE2-L may be administered in combination with one or more additional compounds or therapies.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a transgenic knock-out animal having a modified endogenous RE2-L gene. A transgenic animal can be produced by introducing nucleic acid into the male pronuclei of a fertilized oocyte, e.g., by microinjection, retroviral infection, and allowing the oocyte to develop in a pseudopregnant female foster animal. Still further, the invention contemplates a transgenic animal having an exogenous RE2-L gene generated by introduction of any RE2-L-encoding nucleotide sequence that can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the RE2-L protein to particular cells.

Transgenic animals containing a modified RE2-L gene as described herein are useful to identify RE2-L function. Further, animals containing an exogenous RE2-L gene, e.g., a human RE2-L gene, may be useful in an in vivo context since various physiological factors that are present in vivo and that could effect ligand binding, RE2-L activation, and signal transduction, may not be evident from in vitro cell-free or cell-based assays. Accordingly, it is useful to provide non-human transgenic animals to assay in vivo RE2-L protein function, including ligand interaction, the effect of specific mutant RE2-L proteins on RE2-L protein function and ligand interaction, and the effect of chimeric RE2-L proteins. It is also possible to assess the effect of null mutations, that is, mutations that substantially or completely eliminate one or more RE2-L protein functions.

Transgenic animals containing a modified RE2-L gene as described herein are useful as animal models of anxiety-related disorders, obsessive compulsive behavior or related disorders. The methods of using this model to screen for agents capable of reducing, ameliorating and/or inhibiting psychiatric disorders, motor activity, perseverative or compulsive behaviors, and anxiety, comprises administering a test agent to the animal and determining the ability of the test agent to reduce anxiety or anxiety-related motor activity, or modulate motor activity. The effectiveness of the test agent may be determined by behavioral observation, as described below.

Specific Embodiments

Learning and memory may be evaluated in animals using the Morris Water Maze test of spatial learning. A standard test for anxiety in mice is the elevated plus maze. In these experiments, a modified maze, containing only 2, rather than 4, arms was used. This maze was called the “Hemi-Maze”, because it represents “half” of the elevated plus maze. The “Hemi-Maze” assesses anxiety using the same principle upon which both the elevated plus maze and the light-dark exploration test are based. That is, rodents have two conflicting drives when placed into a novel environment. Rodents are driven to ensure their own safety by remaining in dark, sheltered regions of a novel environment, but are also driven to fully explore their environment, even if that exploration brings them into open, exposed areas of the environment. These mazes have two types of regions, one that is dark and more enclosed, and one that is light and more open. Animals with less anxiety will spend more time in the open regions, and those with greater anxiety will spend more time in the closed regions. The “Hemi-Maze” has two arms, one of which is open and exposed, with sides made of clear Plexiglas, and one of which is more enclosed, with a dark floor and sides painted black. In addition to measuring the proportion of time spent in each side, there are various other measures that can be taken to evaluate different types of anxiety. For instance, time to first cross can be used as a surrogate measure of “freezing,” a rodent behavior undertaken during extreme anxiety. In addition, defecations are counted, because animals tend to defecate more when anxious. Numbers of rears are measured, because anxious animals are hesitant to rear, an action that fully exposes their vulnerable abdomen. In the first couple of minutes in a novel environment, animals will not groom unless engaging in “nervous” motor behavior or compulsive stereotypic motor programs. Therefore, the presence of grooming in the first 2 minutes of the trial is recorded. Finally, the number of crosses is counted, as this can serve both as a measure of general activity, and also as a measure of exploratory drive.

Early grooming is a sign of “nervous” motor activity or compulsive motor activity, such as that observed in anxiety disorders, such as obsessive-compulsive disorder. In obsessive-compulsive disorder the thalamus, cingulate cortex, and striatum have been heavily implicated, consistent with the distribution of RE2-Like by LacZ. RE2-Like null mutants show clear indications of the expression of anxious behaviors including increased guarding (fewer rears) and increased expression of anxiety-related stereotypic motor behaviors.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Generation of RE2-L Knockout Mice

RE2-Like is a receptor identified from genomic DNA. After bioinformatics refinement, the full-length receptor was confirmed by RT-PCR and sequencing as described below.

An extensive database (>4000 sequences) of all known GPCR protein sequences was compiled. The database was expanded by several rounds of homology search, BLASTp BLAST 2.0 was obtained from the NCBI ftp site at ncbi.nlm.nih.gov/ blast/executables. This homology search was performed against public protein sequences from GenBank. The positions of putative transmembrane segments were annotated for each family member using a combination of homology (matching transmembrane positions to those of the closest homologue), hydrophobicity and alignment of key conserved residues to general models (Baldwin et al. (1997) J. Mol. Biol. 272:144-64). In addition to BLAST search, the CLUSTALW algorithm (CLUSTALW 1.7, Nucleic Acids Research, 22(22):4673-4680), which was downloaded from csc.fi/molbio/progs/clustalw/clustalw.html, was also used in some cases to align sequences for annotation of transmembrane regions.

Human RE2-L was initially identified as an open reading frame from a BAC clone. The identified intronless open reading frame was PCR-amplified from genomic. Then a PCR product was produced from cDNA derived from human placental RNA. The resulting PCR fragment was cloned into a series of expression vectors for functional analyses.

### Example 2

### Expression of Human RE2-L

RE2-Like was knocked out using VelociGene™ technology as described in U.S. U.S. Pat. No. 6,856,251, herein specifically incorporated by reference in its entirety. LacZ staining was performed on chimeras and mutant mice. RE2-like maps to a region of the X chromosome frequently associated with X-linked mental retardation syndromes such as Sashi Syndrome, Fragile X Syndrome, and many others. Analysis of expression patterns of RE2-like using a LacZ knock-in revealed an especially intense localization of this gene to septum of the brain. The septum is a structure associated with many functions, most notably learning/memory and emotion.

### Example 3

### Behavioral Testing of RE2-L Knockouts: Learning and Memory

Learning and memory was evaluated in the RE2-like homozygous knockouts (e.g., null mutants) using the Morris Water Maze test of spatial learning. Animals were placed in a 4 foot diameter circular water maze for 2 trial blocks per day every day for 4 days. Submerged just under the water surface was a round escape platform which was hidden from the animals by making the water opaque with non-toxic white paint. Each trial block consisted of three trials separated by 30 second intertribal intervals. Animals were started in a different location of the maze for each trial (8 possible start locations, pseudorandomly assigned). The time taken for the animal to reach the escape platform was measured. If the animal did not locate the escape platform within 1 minute, it was led to the platform by hand. Swim speed was estimated as the mean quadrant crossing time for each animal, and was found not to differ between the two genotypes. For each trial block, the median latency to escape was used for data analysis. After 8 trial blocks, animals were again placed into the water maze for a spatial probe trial. In this trial, the platform was removed, and the swimming pattern of the animals was measured for 30 seconds. The water maze was divided into 4 imaginary quadrants, and the time each animal spent in the quadrant which previously contained the goal platform was compared with time spent in the opposite or adjacent quadrants (adjacent quadrant data expressed as mean time per adjacent quadrant). Animals spending more time in the goal platform in the absence of the tactile cue of the platform exhibited better retention of the location of the goal platform.

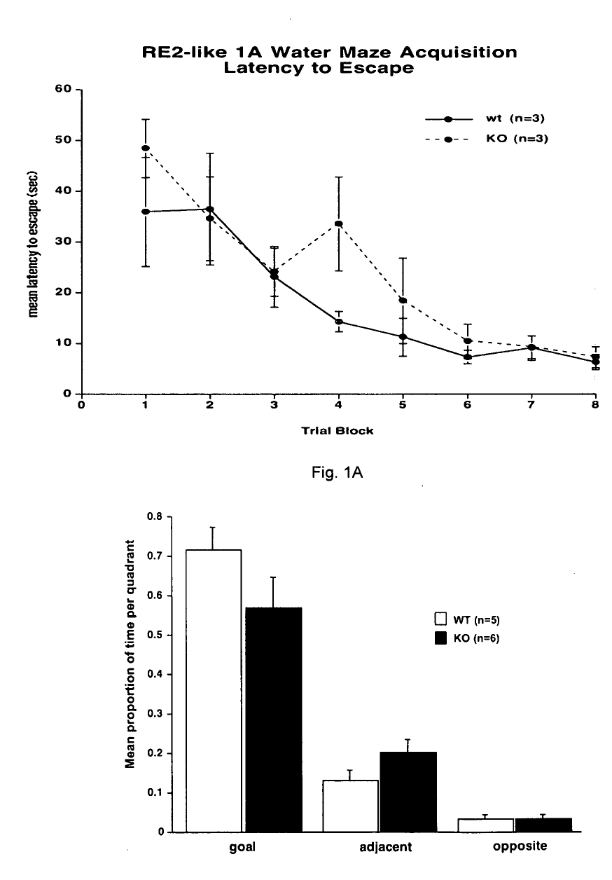

The results are shown in FIGS. 1A-B. RE2-like knock-outs showed no significant delay in learning the location of the escape platform relative to their wild type littermates (FIG. 1A). or in remembering the location once they had learned it (FIG. 1B). These results show that the knockout animals are not afflicted with a mental retardation-like syndrome.

### Example 4

### Behavioral Testing of RE2-L Knockouts: Anxiety Testing

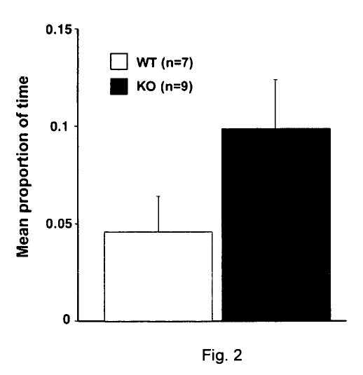

RE2-like knockouts were tested for anxiety on three common tests of anxiety. In all three tests, the mice were given an opportunity to spend time either in a more enclosed, darker location of an apparatus, or a more exposed, lighter region of an apparatus. Rodents feel safer in dark, enclosed places, but the open areas provide a better vantage point for exploration. Therefore, normal rodents will venture out into the open areas, but will spend far more time in the less exposed regions. An increase in open area occupancy is associated either with increased exploratory drive or decreased anxiety. The first task was the Elevated Plus Maze. In this maze, a plus shaped maze is elevated 18 inches from the ground. Two arms of the plus are black with high walls. The other 2 arms are white and have no walls. Animals are placed in the intersection of the plus, and allowed to freely explore for 4 minutes. The second task is the Light-Dark Exploration Task that takes place in a rectangular box divided into two compartments. One compartment is painted black and has a ceiling, while the other compartment is white and has no ceiling. Animals are allowed to explore the chamber freely for 5 minutes. The final task is the Open Field. For this task, animals are placed in a 2 foot by 2 foot field surrounded by a high wall. The field is divided into 9 grids, 8 which lie against the wall and 1 which is located directly beneath a bright light far from any walls. By chance, a mouse should spend 1/9 of the time in each grid. However, normal mice will spend less than this amount of time in the center because it is more exposed. As shown in FIG. 2, RE2-like knockouts spend more time in the open arms of the Elevated Plus Maze (p<0.05 in one cohort, p<0.07 in another).

### Example 5

### Basal Level Activity of RE2-L in HEK293 Cells.

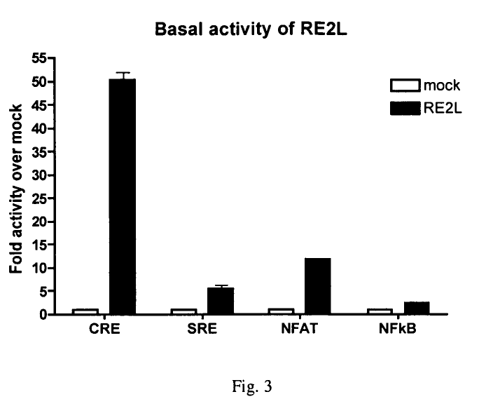

HEK-293 hz cells were transfected with human RE2L cDNA cloned in pcDNA3.1 along with the luciferase reporter plasmids CRE-luciferase, SRE-luciferase, NFAT-luciferase and NFkB-luciferase. The basal signaling activity of hRE2L was tested with each of these reporters by performing a luciferase activity assay 48 hours after transfection. The results are shown in FIG. 3. hRE2L has a high basal activity and caused a 50-fold increase in the CRE-luciferase activity suggesting that activation of hRE2L probably leads to an increase in intracellular cyclic-AMP (cAMP). Transfection of hRE2L also caused a 12-fold increase in NFAT-luciferase activity, a 5-fold increase in SRE-luc activity and a 2-fold increase in NF-kB-luciferase activity.

What is claimed is:

1. A method of identifying an agent capable of modulating RE2-L protein activity or expression, comprising
(a) contacting a test agent with a RE2-L protein; and
(b) determining the ability of the test agent to modulate RE2-L activity or expression.

2. The method of claim 1, wherein the agent is an inhibitor of RE2-L activity or expression.

3. The method of claim 2, wherein the agent is an inhibitor of RE2-L expression.

4. The method of claim 3, wherein the agent is a RE2-L antisense molecule.

5. The method of claim 3, wherein the agent is a siRNA molecule capable of interfering with the expression of the gene encoding RE2-L.

6. The method of claim 2, wherein the agent is an inhibitor of RE2-L activity.

7. The method of claim 6, wherein the agent is a blocking antibody.

8. The method of claim 6, wherein RE2-L activity is measured by measuring cAMP levels.

9. The method of claim 6, wherein RE2-L activity is measured using a reporter molecule.

10. The method of claim 9, wherein the reporter molecule is a CRE, NFAT, SRE, or NF-κB reporter molecule.

11. An in vivo method for identifying an agent capable of modulating a RE2-L protein, comprising:
(a) administering a test agent to an animal expressing a RE2-L protein; and
(b) determining the ability of the test agent to modulate RE2-L.

12. The method of claim 11, wherein the ability of the test agent to modulate RE2-L is determined by measuring behavior of psychiatric disorders.

13. The method of claim 11, wherein the psychiatric disorders are measured by a test selected from the group consisting of elevated plus maze, open field testing, light-dark exploration tests, tests of social interation, sensory tests, sensory gating tests, quantification of animal freezing, defecations, rears and early grooming.

14. A non-human animal comprising an altered or deleted RE2-L gene.

15. The non-human animal of claim 14, characterized by exhibiting anxiety-related disorders, obsessive, perseverative, or compulsive behavior or related disorders.

16. The non-human animal of claim 14, characterized by exhibiting pervasive developmental disorders.

17. The non-human animal of claim 14, selected from the group consisting of a mouse, a rat, a rabbit, a guinea pig, a hamster, a cat, a dog, and a sheep.

18. The non-human mammal of claim 14, further comprising a human OPSDL3 transgene.


SEQUENCE LISTING (2 sequences)

SEQ ID NO: 1 - DNA, 1527 bp, Homo sapien
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 2 - protein, 508 aa, Homo sapien
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP